(19) United States
(12) Patent Application Publication
(10) Pub. No.: US 2022/0024957 A1
(43) Pub. Date: Jan. 27, 2022

(54) ANTICANCER ACTIVITIES OF A NOVEL FAMILY OF ETHACRYNIC ACID DERIVATIVES

(71) Applicants: UNIVERSITE EUROMEDITERRANEENNE DE FES, Fès (MA); UNIVERSITE SULTAN MOULAY SLIMANE BENI MELLAL, Beni Mellal (MA)

(72) Inventors: Saïd EL KAZZOULI, Fès (MA); Abdelmadjid ZYAD, Beni Mellal (MA); Nabil EL BRAHMI, Fès (MA); Abdelmoula EL ABBOUCHI, Fès (MA); Khalid BOUJDI, Fès (MA); Mostapha BOUSMINA, Fès (MA); Hassan AIT MOUSE, Beni Mellal (MA); Mounir TILAOUI, Beni Mellal (MA)

(21) Appl. No.: 17/297,252

(22) PCT Filed: Nov. 29, 2019
(86) PCT No.: PCT/MA2019/000013

§ 371 Date: May 26, 2021

(30) Foreign Application Priority Data
Nov. 29, 2018 (MA) 44038

(51) Int. Cl. C07F 9/6509; A61P 35/00

(52) U.S. Cl. CPC C07F 9/650952; A61P 35/00

(57) ABSTRACT

A new class of small anti-cancer molecules derived from ethacrynic acid (symbolized by AE) is presented. AE analogues are synthesized and then the in vitro cytotoxic activities thereof are evaluated on the P815 tumour cell line using the MIT test. The AE derivative which exhibited the best in vitro cytotoxicity is then tested in vivo using the DBA2/P815 (H₂d) mouse model. At 30 mg/kg, the effective dose, the animals showed general tolerance with a percentage survival of around 80%, and no significant weight loss is observed.

### FIELD OF THE INVENTION

The present invention relates to a novel class of small anticarcinogenic molecules derived from ethacrynic acid (symbolised by EA). The invention relates to the in vitro and in vivo anticancer activities and the methods for preparing the novel family of EA. Novel analogues of EA were synthesised then, their in vitro cytotoxic activities were evaluated on the P815 tumour cell line using the MTT test. The EA derivative, which exhibited the best in vitro cytotoxicity, was next tested in vivo using the DBA2/P815 (H₂d) mouse model. At 30 mg/kg, the effective dose, the animals showed a general tolerance with a survival percentage in the region of 80%, and no significant loss of weight was observed.

### PRIOR ART

Remarkable advances have been made in the chemotherapy field and this has been through the introduction of novel molecules such as thalidomide, lenalidomide and bortezomib. Despite this, cancer still remains an incurable disease. The efficacity of chemotherapy still needs to be improved and for this to be so while reducing the toxicity and the secondary effects of treatments. In addition, the intrinsic or acquired resistance of a large number of tumours to chemotherapy is also a major obstacle facing the efficacity of anticancer treatments. Several mechanisms for cell resistance to different active substances have been identified (Moscow and Cowan 1988). Taking this into consideration, the search for novel agents efficacious in chemotherapy capable of treating the different types of cancer is still indispensable.

According to the World Health Organisation, cancer is one of the main causes of mortality in the world. Cancers of the lung, the liver, the stomach, the colon and the breast are the most widespread in the world. In view of this diversity of the types of cancer, the development of novel anticancer agents takes a very important place in the oncology field. In addition, the development of specific molecules for the combat against this disease while circumventing the obstacle of cell resistance is necessary.

Microsomal glutathione S-transferase 1 (mGST1) and glutathione S-transferase pi (GSTpi) are often overexpressed in tumours thereby conferring resistance to a certain number of chemotherapeutic agents, such as cisplatin and doxorubicin (DOX) [(Johansson et al. 2011)]. These enzymes catalyse the conjugation of glutathione and act as detoxification enzymes.

EA or 2,3-dichloro-4-(2-methylenebutryl)-phenoxyacetic acid, which is a well-known diuretic, is used in the treatment of hypertension and swelling caused by diseases such as congestive cardiac insufficiency, hepatic insufficiency and renal insufficiency [(Borne, Levi, and Wilson 2002; Koechel 1981)]. It is also known as a good inhibitor of class pi glutathione S-transferase. EA has an acid function and an α,β-unsaturated carbonyl unit which reacts with nucleophiles, such as the thiol of glutathione S-transferase P1-1 (GSTP1-1, GSTpi). In addition, it has recently been confirmed that EA inhibits the signalling of Wnt/beta catenin which plays an important role in the regulation of cell proliferation, differentiation and apoptosis [(Liu et al. 2006; Lu et al. 2009; Janovská and Bryja 2017)].

In order to improve the capacity of EA to inhibit the growth of cancer cells in vivo while conserving its good inhibition activity of glutathione S-transferase, we propose in this invention a novel synthesis of powerful and original anticancer agents. Thus, on the basis of our very encouraging results concerning the antitumour activities of various analogues of EA in vitro on a panel of cell lines [(El Brahmi et al. Nanoscales, 2015; Mignani et al. Eur. J. Med. Chem. 2016)], we propose in this invention, the synthesis and the in vivo evaluation of the best analogues by making structural modifications, on the basic backbone of the EA molecule. These chemical transformations result in the formation of amide bonds between the carboxylic acid function of EA and primary and secondary amines. The acrylate part has, for its part, remained intact.

### BRIEF DESCRIPTION OF THE FIGURES

Diagram 1. Synthesis of EA derivatives: P3, P4 and P5.

FIG. 1. Cytotoxic activity of EA derivatives (P3, P4 and P5) against the P815 tumour line.

Table 1 IC₅₀ values of the compounds P3, P4 and P5.

FIG. 2. Effect of the molecule P4 on tumoral evolution in tumour bearing DBA2 mice.

FIG. 3. Evolution of body weight of DBA2 mice treated with the compound P4.

FIG. 4. Curves of monitoring mice treated with the compound P4.

### DESCRIPTION OF THE INVENTION

Synthesis of Target Molecules

The EA derivatives are prepared from commercially available EA (diagram 1). The treatment of EA under the conditions of a peptide reaction by different amines in a DCM/DMF mixture in the presence of 1-ethyl-3-(3-dimethylaminopropyl)carbodiimide (EDCI) and 4-dimethylaminopyridine (DMAP) at room temperature provides the desired products with moderate yields.

The presence of the phenol function on the modified EA was used for the nucleophilic substitution with chlorophosphates. Thus, treatment with different chlorophosphates in dichloromethane (DCM) in the presence of trimethylamine as organic base makes it possible to obtain the desired compounds with moderate yields.

General method for the preparation of the molecules P3 and P4. To a mixture of EDCI (1.2 equiv.), DMAP (in catalytic quantity) and 1 equivalent of EA in anhydrous DMF (5 mL), 1 equivalent of amines (4-hydroxyphenyl piperazine or 4-methoxyphenyl piperazine) is added at 0° C. The reaction mixture is stirred overnight at room temperature, then, ethyl acetate (100 mL) is added and the organic phase is washed with water (2×50 mL) and brine (3×50 mL), dried over anhydrous MgSO₄ and concentrated using a rotatory evaporator. The residue obtained is purified by flash chromatography.

P3. Yield=60%. (DCM/EtOAc (9:1 to 8:2 (v/v))). ¹H NMR (CDCl₃; 400 MHz), δ (ppm): 7.16 (d, J=8.6 Hz, 1H, Hₐᵣ), 7.00 (d, J=8.6 Hz, 1H, Hₐᵣ), 6.84 (d, J=9.0 Hz, 2H), 6.79 (d, J=9.0 Hz, 2H), 5.95 (s, 1H), 5.61 (s, 1H), 5.54 (s, 1H), 4.88 (s, 2H), 3.76-3.84 (m, 4H), 3.06-3.12 (m, 2H), 2.99-3.06 (m, 2H), 2.48 (q, J=7.4 Hz, 2H), 1.16 (t, J=7.4 Hz, 3H). ¹³C NMR (CDCl₃, 101 MHz); δ (ppm): 195.8 (C, C(=0)), 165.3 (C, C(═O)N), 155.2 (C, Cₐᵣ), 150.7 (C, Cₐᵣ), 150.2 (C, Cₐᵣ), 144.9 (C, Cₐᵣ), 133.8 (C, Cₐᵣ), 131.4 (C, Cₐᵣ), 128.8 (CH₂, C═CH₂), 127.1 (2CH, CHₐᵣ), 122.8 (C, Cₐᵣ), 119.3 (CH, CHₐᵣ), 116.0 (2CH, CHₐᵣ), 110.37 (CH, CHₐᵣ), 68.7 (CH₂), 51.5 (CH₂), 50.7 (CH₂), 45.7 (CH₂), 42.4 (CH₂), 23.4 (CH₂), 12.4 (CH₃). HRMS (+ESI) m/z: [M+H]⁺ calculated for C₂₃H₂₄Cl₂N₂O₄: 463.1188, found, 463.1192. IR (neat): v=3325 (OH), 1654 (C=0), 1645 (C═C) cm⁻¹.

P4. Yield=47%. (DCM/EtOAc (8:2 (v/v))). ¹H NMR (CDCl₃; 400 MHz); δ (ppm): 7.15 (d, J=8.5 Hz, 1H, Hₐᵣ), 6.99 (d, J=8.5 Hz, 1H, Hₐᵣ), 6.93-6.82 (m, 4H, Hₐᵣ), 5.93 (s, 1H), 5.59 (s, 1H), 4.86 (s, 2H), 3.83-3.73 (m, 7H), 3.08 (t, J=5.0 Hz, 2H), 3.03 (t, J=5.0 Hz, 2H), 2.46 (q, J=7.4 Hz, 2H), 1.14 (t, J=7.4 Hz, 3H). ¹³C NMR (CDCl₃; 101 MHz); δ (ppm): 195.9 (C, C(=0)), 165.3 (C, C(═O)N), 155.4 (C, Cₐᵣ), 154.7 (C, Cₐᵣ), 150.3 (C, Cₐᵣ), 145.2 (C, Cₐᵣ), 133.9 (C, Cₐᵣ), 131.7 (C, Cₐᵣ), 128.8 (CH₂, C═CH₂), 127.3 (2CH, CHₐᵣ), 123.0 (C, Cₐᵣ), 119.3 (CH, CHₐᵣ), 114.7 (2CH, CHₐᵣ), 110.9 (CH, CHₐᵣ), 68.9 (CH₂), 55.7 (OCH₃), 51.6 (CH₂), 51.0 (CH₂), 45.8 (CH₂), 42.6 (CH₂), 23.7 (CH₂), 12.6 (CH₃).

HRMS (+ESI) m/z: [M+H]⁺ calculated for C₂₄H₂₆Cl₂N₂O₄: 477.1348, found, 477.1324. IR (neat): v=1661 (C=0) cm⁻¹. Elementary analyses for C₂₄H₂₆Cl₂N₂O₄; calculated: C, 60.38; H, 5.49; N, 5.85, found: C, 60.42; H, 5.24; N, 5.69.

P5. To a mixture of P3 (1 equiv.) and triethylamine (1.1 equiv.) in anhydrous DCM at 0° C., diethyl chlorophosphate is added drop by drop (1 equiv.). The reaction mixture is stirred at room temperature overnight. After, DCM (20 mL) is added and the organic phase is washed with water (10 mL) and brine (10 mL), dried over anhydrous MgSO₄ and then concentrated using a rotary evaporator. The crude product is purified by flash chromatography. Yield=46%. (DCM/EtOAc (2:1 to 1:1 (v/v))). ³¹P NMR (CDCl₃; 162 MHz), δ (ppm): −5.8 (s, P). ¹H NMR (CDCl₃; 400 MHz), δ (ppm): 7.18-7.09 (m, 3H, Hₐᵣ), 6.98 (d, J=8.6 Hz, 1H, Hₐᵣ), 6.88-6.82 (m, 2H, Hₐᵣ), 5.93 (t, J=1.5 Hz, 1H), 5.58 (s, 1H), 4.86 (s, 2H), 4.28-4.10 (m, 4H), 3.82-3.74 (m, 4H), 3.14 (t, J=5.1 Hz, 2H), 3.09 (t, J=5.1 Hz, 2H), 2.45 (q, J=7.4 Hz, 2H), 1.34 (td, J=7.1, 1.0 Hz, 6H), 1.13 (t, J=7.4 Hz, 3H). ¹³C NMR (CDCl₃; 101 MHz), δ (ppm): 195.9 (C, C=0), 165.3 (C, C(═O)N), 155.3 (C, Cₐᵣ), 150.3 (C, Cₐᵣ), 148.2 (C, Cₐᵣ), 144.9 (d, J=7.0 Hz, C, Cₐᵣ), 133.9 (C, Cₐᵣ), 131.6 (C, Cₐᵣ), 128.8 (CH₂, C═CH₂), 127.2 (CH, Cₐᵣ), 122.9 (C, Cₐᵣ), 120.8 (d, J=4.7 Hz, 2CH, Cₐᵣ), 118.2 (2CH, Cₐᵣ), 110.8 (CH, Cₐᵣ), 68.9 (CH₂, OCH₂), 64.6 (d, J=6.1 Hz, 2CH₂), 50.7 (CH₂), 50.0 (CH₂), 45.6 (CH₂), 42.3 (CH₂), 23.5 (CH₂), 16.2 (d, J=6.7 Hz, 2CH₃), 12.5 (CH₃). HRMS (+ESI) m/z: [M+H]⁺ calculated for C₂₇H₂₃Cl₂N₂O₇P: 599.1481, found, 599.1486. IR(neat): v=1663 (C=0) cm⁻¹.

Antitumour Activity

The P815 tumour line of the murine mastocytoma (ATCC: TIB64) used in this study was graciously supplied to our laboratory by Dr. Michel Lepoivre, UMR CNRS 9198, Bát. 430, Université de Paris-Saclay, France. This line is maintained in culture in complete DMEM (Dulbecco's Modified Eagle's Medium) supplemented with 5% FBS (Gibco BRL, CergyPontoise, France), 100 IU/mL of penicillin, 100 μg/mL of streptomycin and 0.2% of sodium bicarbonate (Sigma) in a humid atmosphere at 37° C. and 5% CO₂.

Cytotoxicity Test

Before carrying out the cytotoxicity test, the viable cells are counted by trypan blue exclusion assay. The aim being to obtain a suspension of 4×10⁴ cells/mL to incubate in 100 μL of complete culture medium by flat bottomed wells of 96-well microculture plates [(Bioster, Bastia di Rovolon, Italy)]. The micro-culture thereby obtained is incubated for 24 hours before carrying out the cytotoxicity tests. The latter are then carried out by applying decreasing doses of the molecules (P3, P4 and P5) obtained by half into half dilutions, in 100 μL of DMEM medium. Each test is carried out in duplicate and repeated three times with positive and negative controls. The three molecules P3, P4 and P5 are firstly solubilised in DMSO of which the final concentration, during the test, will not exceed 0.5% (this concentration having no effect on cell growth). These micro-cultures are incubated at 37° C. in humid atmosphere containing 5% CO₂ for 48 h.

The determination of cytotoxic activity is carried out by evaluating the concentration of tested molecules inhibiting 50% of cell growth (IC₅₀) compared to a control cultured in the same conditions in the absence of the studied compound. This simple and rapid test enables a rapid selection to be made of molecules exhibiting activity capable of limiting or stopping the growth of cancer cells. The revelation of the cytotoxic activity is performed using the MTT test: 3-(4,5-dimethylthiazol-2-yl)-2,5-diphenyl tetrazolium bromide [(Mosmann 1983; Tilaoui et al. 2015)]. This test is carried out as described and modified by Mosmann, 1983. After 48 h incubation in the culture conditions cited below, 20 μL of a solution of MTT (5 mg/mL of PBS) is added. After 4 hours of incubation under the same culture conditions, the violet crystals formed further to the reduction of MTT by the mitochondrial dehydrogenases of living cells are solubilised by adding 100 μL of a solution if HCl/isopropanol (24:1). The optical density (OD) is next read at two wavelengths, 540 nm and 630 nm, using a MultisKan EX microplate spectrophotometric reader. Thus, the effect of P3, P4 and P5 on cell viability may be measured by using the following formula: Cell viability (%)=(OD_{molecules}/OD_{control})×100

With:

OD: optical density corresponding to cells treated with the molecules (P3, P4 and P5) and/or Methotrexate MTX (used as positive control).

OD_{con}: optical density corresponding to the negative control (non-treated cells).

In Vivo Antitumour Activity

The model chosen for this study is constituted of the P815 tumour line and the DBA2 (H2^{d}) syngeneic mouse strain purchased from the Orleans breeding centre in France). P815 cells are capable of inducing solid tumours in DBA2 mice. The DBA2 mice are raised in an animal facility at a temperature of 25° C. and a photoperiod of 12 hours. Food and drink are supplied ad libitum to the animals. The mice used in our experiments are seven to eight weeks old with body weights comprised between 20 and 24 g. Sex is not taken into consideration in our tests.

Induction of Primary Tumours

P815 cancer cells are collected by centrifugation at 1400 rpm for 10 min at room temperature. The centrifugation pellets obtained are washed twice with PBS and resuspended in 1 mL of PBS and counted. Around 10⁷ living cells in suspension in a volume of 100 μL of PBS are injected, under ether anaesthesia, by sub-cutaneous route into the dorso-lumbar region of each mouse. A small tumour appears at the injection site at the end of one week to 10 days.

Treatment of the Mice

When the tumours become palpable, the mice are split up into 4 lots with 6 mice per lot (day 0). Next, every two days, the mice are treated by force-feeding in the following manner:

The mice of lots A (control), B, C and D receive respectively 100 μL of vegetable oil alone, 10, 20 and 30 mg/kg of the molecule P4 (the most cytotoxic compared to the other molecules tested) dissolved in 100 μL of vegetable oil. The treatment is carried out at a rate of a single oral administration every 48 h for 14 days. The weight and the survival of the mice, as well as tumour volume, are measured every two days for 28 days.

The tumour volume on day n (TVn) is calculated in the following manner:

TV=(L*W)/2 where L and W represent the length and the width of the tumour, as described by Yoshikawa [(Yoshikawa et al. 1995)].

Statistical Analyses

The experiments conducted in vitro were carried out in triplicate. The data reflect the average of three different experiments. The statistical analysis for these studies uses a Student Test. The data are considered as statistically significative for p<0.05. Concerning the in vivo study, each condition comprises 6 mice, n=3, and the statistical analysis was carried out using the one-way ANOVA test followed by the Tukey and Scheffé post hoc test. The data are considered as statistically significative for p<0.05.

Results

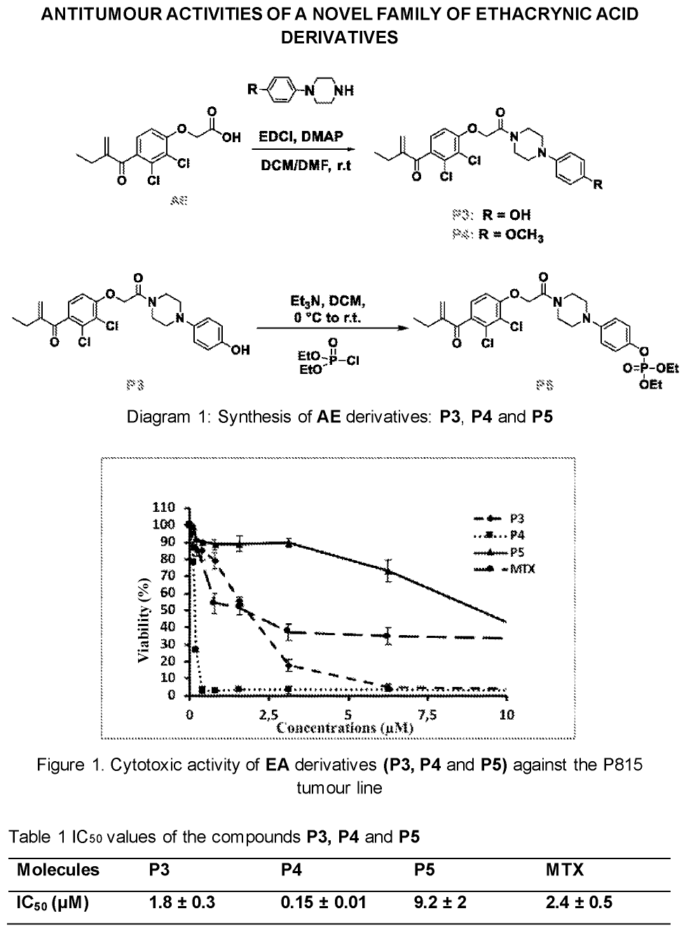

The in vitro cytotoxic activity was measured by the MTT test against the P815 tumour line (FIG. 1). This cytotoxicity starts at low concentrations and increases in a dose dependent manner for all the molecules tested. The latter exhibit a very high cytotoxic activity with an IC₅₀ comprised between 0.15 and 9.2 μM (Table 1), and it is the molecule P4 that showed a very strong cytotoxic effect compared to the other molecules P3 and P4 with an IC₅₀=0.15 μM. Thus, the compound P4 was evaluated for its antitumour effect.

Antitumour Activity: Preclinical Studies

In vivo tests represent an important step in the study of the antitumour activity of our molecule. The objective is to pass to a test in conditions that come closest to the reality of the disease. To this end, DBA-2 (H2^{d}) mice bearing P815 solid tumours were used with the aim of testing the in vivo antitumour effect of the molecule P4, which showed very significant cytotoxic activity compared to the other molecules tested P1 and P3.

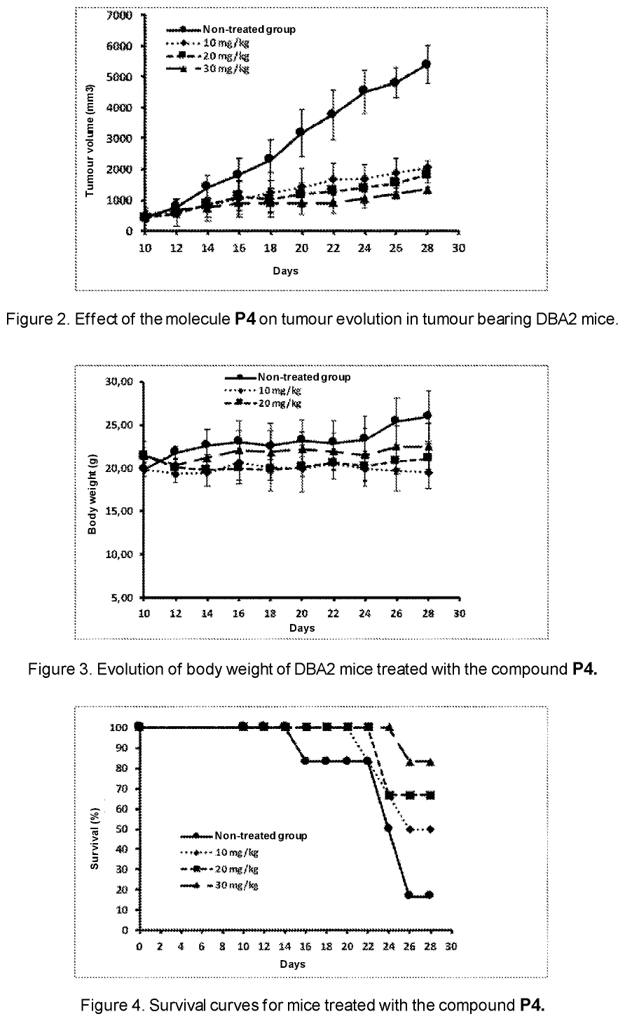

The tests were carried out by oral administration (force-feeding) of the molecules dissolved in a vegetable oil (edible oil) to mice aged 6-8 weeks every 48 hours for a duration of 14 days. The results obtained are presented in FIG. 2. On reading this figure, the treatment of mice by the molecule P4 induced a significant decrease in tumour volume. The administration by force-feeding of the molecule P4 at doses of 10, 20 and 30 mg/kg induced a significant reduction in tumour volume after 28 days of treatment compared to the control mice (FIG. 2). Moreover, no significative difference was observed between the doses used. However, it was noted that the mice treated with the 30 mg/kg dose showed tolerance vis-à-vis this dose with a survival rate of around 80% (FIG. 4), and no significative impact on the loss of body weight (FIG. 3) compared to mice treated with 10 and 20 mg/kg doses (P<0.05). Also, no significative difference is noted in the decrease in tumour volume after treatment by the three doses (10, 20 and 30 mg/kg) (P<0.05). Thus, we can consider from an efficacity viewpoint that the 30 mg/kg dose of the compound P4 is more efficacious compared to the other doses tested.

1. A synthesis method for synthesising novel amide derivatives of ethacrynic acid (EA) from commercially available EA and different primary and secondary amines as starting precursors, comprising the following steps:
a) reaction of ethacrynic acid EA with the amines in a mixture of dichloromethane (DCM) and N,N-dimethylformamide (DMF) in the presence of 1-ethyl-3-(3-dimethylaminopropyl)carbodiimide (EDCI) and 4-dimethylaminopyridine (DMAP) to obtain compounds P3 of formula R=OH or P4 of formula R=OMe: 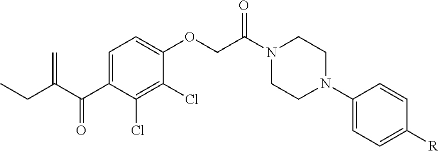
b) the reaction between diethyl chlorophosphate and the compound P3 (R=OH), formed at step (a) in dichloromethane (DCM) at room temperature in the presence of triethylamine results in the compound of formula P5

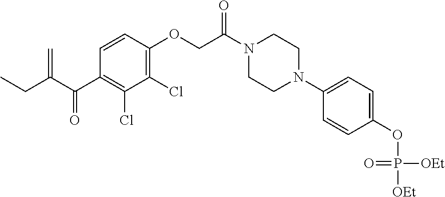 [SMILES: C=C(CC)C(=O)c1ccc(OCC(=O)N2CCN(c3ccc(OP(=O)(OCC)OCC)cc3)CC2)c(Cl)c1Cl]

2. The synthesis method according to claim 1, resulting in the amide derivatives of ethacrynic acid with the groups 4-(piperazine-1-yl)phenol for the compound P3, 1-(4-methoxyphenyl)piperazine for the compound P4 and diethyl (4-(piperazine-1-yl)phenyl) phosphate for the compound P5.

3. A method for inhibiting or treating cancer in vitro with the compounds of claim 2.

4. The method according to claim 3, wherein the cancer is a P815 cancer line.

5. The method according to claim 4, comprising testing the compound P4 in preclinical studies on a mouse model, wherein the mouse model is constituted of P815 tumour line and DBA2 (H2^{d}) syngeneic mouse strain.

6. The method according to claim 5, wherein the testing comprises testing at least three doses of the compound P4 to evaluate an evolution of tumour volume in tumour bearing DBA2 mice, the at least three doses including 10 mg/kg, 20 mg/kg and 30 mg/kg.

7. The method according to claim 6, wherein the at least three doses are tested to evaluate an evolution of body weight of DBA2 mice treated with the compound P4.

8. The method according to claim 7, wherein the at least three doses are tested to evaluate the survival of mice treated with the compound P4.

9. The method according to claim 8, wherein the at least three doses are tested to evaluate an effective dose of the compound P4.

10. The method according to claim 9, wherein the effective dose is the 30 mg/kg dose so that the mice treated with the 30 mg/kg dose of the compound P4 show a tolerance vis-à-vis the 30 mg/kg dose with a survival rate of around 80% and no significant impact on a loss of body weight.
